(19) United States
(12) Patent Application Publication
(10) Pub. No.: US 2024/0279137 A1
(43) Pub. Date: Aug. 22, 2024

(54) CATALYTIC FORMIC ACID AND HYDROGEN PRODUCTION

(71) Applicant: University of Massachusetts, Boston, MA (US)

(72) Inventors: David K. Ryan, Reading, MA (US); Elizabeth G. Farrell, Salem, MA (US)

(21) Appl. No.: 18/570,724

(22) PCT Filed: Jun. 16, 2022
(86) PCT No.: PCT/US2022/072984

§ 371 Date: Dec. 15, 2023

Related U.S. Application Data
(60) Provisional application No. 63/202,618, filed on Jun. 17, 2021.

(51) Int. Cl. C07C 1/12; C01B 3/38; C07C 53/02

(52) U.S. Cl. CPC C07C 1/12; C01B 3/38; C07C 53/02; C01B 2203/1235; C01P 2006/80; C07C 2523/00

(57) ABSTRACT

The present disclosure presents a method of producing formic acid, hydrogen, or a mixture thereof. The method includes forming a reaction mixture. The reaction a mixture is passed over or placed in contact with a metal catalyst component comprising a nanostructured surface, in contact with water, and carbon dioxide. The reaction mixture reacts to produce a reaction product comprising formic acid, hydrogen, or a mixture thereof.

### CROSS-REFERENCE TO RELATED APPLICATION

This application claims the benefit of priority to U.S. Provisional patent application Ser. No. 63/202,618 entitled “CATALYTIC FORMIC ACID AND HYDROGEN PRODUCTION,” filed Jun. 17, 2021, the disclosure of which is incorporated herein in its entirety by reference.

### BACKGROUND

Hydrogen is considered to present opportunities for environmentally friendly energy production. One of the challenges associated with using hydrogen as an energy source is the efficient production of hydrogen. It would be desirable to establish simple protocols for the production of acceptably pure hydrogen.

### SUMMARY OF THE DISCLOSURE

The present disclosure presents a method of producing formic acid, hydrogen, or a mixture thereof. The method includes forming a reaction mixture. The reaction mixture is passed over or placed in contact with a metal catalyst comprising a nanostructured surface in contact with water, and carbon dioxide. The carbon dioxide may also be present in an aqueous form and include carbonic acid, bicarbonate, and carbonate. The reaction mixture reacts to produce a reaction product comprising formic acid, hydrogen, or a mixture thereof.

There are several advantages associated with various embodiments of the method and kit of the present disclosure, some of which are unexpected. For example, according to various embodiments of the present disclosure, hydrogen gas of high purity can be obtained. Additionally, according to various embodiments of the present disclosure, a metal or metal compound with a synthesized nanostructured surface can be an important component for hydrogen production. Other advantages, according to various embodiments of the present disclosure include a lack of any carbon monoxide (CO) produced that might act as a poison if the hydrogen is used in fuel cells. According to various embodiments of the present disclosure, there is potential for continuous production of hydrogen with a flow through reactor. According to various embodiments of the present disclosure, a method or system including the metal catalyst catalyzes a reaction using commonly available feedstocks (e.g., water, carbon dioxide, nickel, carbonate, bicarbonate salts, and cobalt) and is substantially free of highly toxic materials and does not produce toxic waste materials. According to various embodiments of the present disclosure, systems and methods using the metal catalyst do not require high temperatures or pressures. According to various embodiments of the present disclosure cobalt and/or nickel can be recycled for reuse in the reaction system. Other advantages, according to various aspects of the present disclosure, can include the ability to make formic acid that can effectively store hydrogen—that is the formic acid can be degraded by the metal catalyst particles described herein to form hydrogen.

### BRIEF DESCRIPTION OF THE FIGURES

The drawings illustrate generally, by way of example, but not by way of limitation, various embodiments discussed in the present document.

FIG. 1A shows scanning electron microscopy (SEM) images of a nickel metal catalyst particle.

FIG. 1B shows scanning electron microscopy (SEM) images of a cobalt metal catalyst particle.

FIG. 2 is a graph showing formic acid degradation vs. argon pressure over a nanostructured cobalt (n-Co) catalyst.

FIG. 3 is a schematic diagram of an assembly for forming formic acid.

FIG. 4 is a schematic diagram of an assembly for forming formic acid and hydrogen.

FIG. 5 is a schematic diagram of another assembly for forming formic acid and hydrogen.

FIG. 6 is a dual chamber device.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

In the search for sustainable cleaner energy sources, hydrogen is considered as an intriguing future energy source, overcoming the environmental impact and sustainability issues associated with fossil fuels.

The process described here is not water splitting, but rather produces hydrogen by first generating formic acid/formate catalytically from water and the carbon dioxide/carbonate system over a nanostructured metal catalyst. The formic acid/formate produced is readily degraded to carbon dioxide and hydrogen catalytically either in the same system (e.g., left in contact with the catalyst) or a separate reaction chamber. Producing hydrogen in this way requires less energy and can be accomplished with minimal thermal input. In some further embodiments, it is possible to use light to enhance the reaction (e.g., to produce more formic acid/formate). Besides directly adding heat to drive the reaction another way to increase the temperature is to expose the reaction vessel to a directional magnetic field, an alternating magnetic field, or both. If the catalyst is ferromagnetic, an alternating magnetic field will generate heat through induction. Typically, formate (the deprotonated form of formic acid) is the dominate species at the optimal pH of 6.1, but product deprotonation will be dependent on the pH of the system.

This disclosure can provide a system or method for producing high formic acid and hydrogen levels. In some examples, high amounts of formic acid produced at a rate in a range of at least about 0.10 μmol/(grams of catalyst—hour) at least about 50 μmol/(grams of catalyst—hour) or 0.5 mol/(grams of catalyst—hour), or even higher. H₂ can be produced in high amounts at a rate in a range from about 0.04 μmol/(grams of catalyst—hour) to about 40 μmol/(grams of catalyst—hour). Hence, the disclosed reaction setup is useful for generating hydrogen to feed a fuel cell for the production of cleaner energy with a sustainable energy source. Additionally, the absence of CO production with produced H₂ can be beneficial since even a small amount of CO present in the H₂ stream (few parts per million) can lead to the poisoning of Pt anodes in fuel cells causing a large loss of efficiency.

According to various embodiments of the present disclosure, a method of producing hydrogen includes disposing a metal catalyst component exhibiting a nanostructured surface, a quantity of carbon dioxide, and water in a reaction vessel in the presence or absence of light. The light can include natural sunlight or a source of artificial light. Once the components are disposed in the reaction vessel, the temperature of the reaction vessel is increased. For example, the temperature can be in a range of from about 10° C. to about 150° C. The reaction vessel can be a container that is configured to allow transmission of light into the reaction vessel. In some aspects, the reaction vessel can be located proximate to a source of artificial light or in sunlight. The metal catalyst component acts as a catalyst to produce formic acid from the carbon dioxide and water.

The quantity of the metal catalyst component in the reaction vessel can be in a range from about 1 mg to about 100 kg. However, the amount of the metal catalyst component can be any suitable amount for production of different quantities of formic acid and/or H₂. The metal catalyst component can include an elemental metal or alloy thereof. In some examples the metal catalyst component can include a metal oxide. The elemental metal or alloy thereof can be in a range from about 0.01 wt % to about 100 wt % of the metal catalyst component, about 80 wt % to about 100 wt %, 95 wt % to about 100 wt %, or less than, equal to, or greater than about 0.01 wt %, 0.5, 1, 10, 20, 30, 40, 50, 60, 70, 75, 80, 85, 90, 95, or 100 wt %.

The metal catalyst component can include one or more metals or alloys thereof. For example, the metal catalyst component can include at least one of the elements Co, Ti, Ru, Rh, Pd, Os, Ir, La, Ce, Fe, Cu, and Ni or an alloy thereof. In various embodiments the metal catalyst component can include substantially 100 wt % cobalt, nickel, or an alloy thereof. In some examples, the catalyst can include a support structure. For example, the catalyst component can include any of the aforementioned metal catalysts placed on a support structure such as an activated carbon support or an aluminum oxide support.

The metal catalyst component can act to initiate and maintain the formation of the formic acid, hydrogen, or both in some embodiments. Without intending to be bound to any theories, the inventors believe that the formation of the nanostructured surface on the metal catalyst component contributes to the formation of the hydrogen. The nanostructured surface can have a plurality of features having any suitable configuration. For example, each of the plurality of features of the nanostructured surface can have a nanoleaf configuration. Each of the features can have different nanoscale dimensions. For example, each of the nanostructures can have the following dimensions: about 1 nm to about 1,000 nm in length, about 1 nm to about 1,000 nm in height, and about 1 nm to about 50 nm in thickness.

The metal catalyst component can be synthesized through a chemical process. This is a so called “bottom up” approach in that the nanostructured surface is formed through a chemical process building nanomaterials from molecules, ions or metal atoms. This is directly contradictory to other examples of metal catalyst components, which include nanostructured surfaces formed through chemical or laser etching techniques-so called “top down” approaches. Examples of metal catalyst components made through top-down approaches are described in United States Published Patent Application No. 2012/0097521 to (“Shen”).

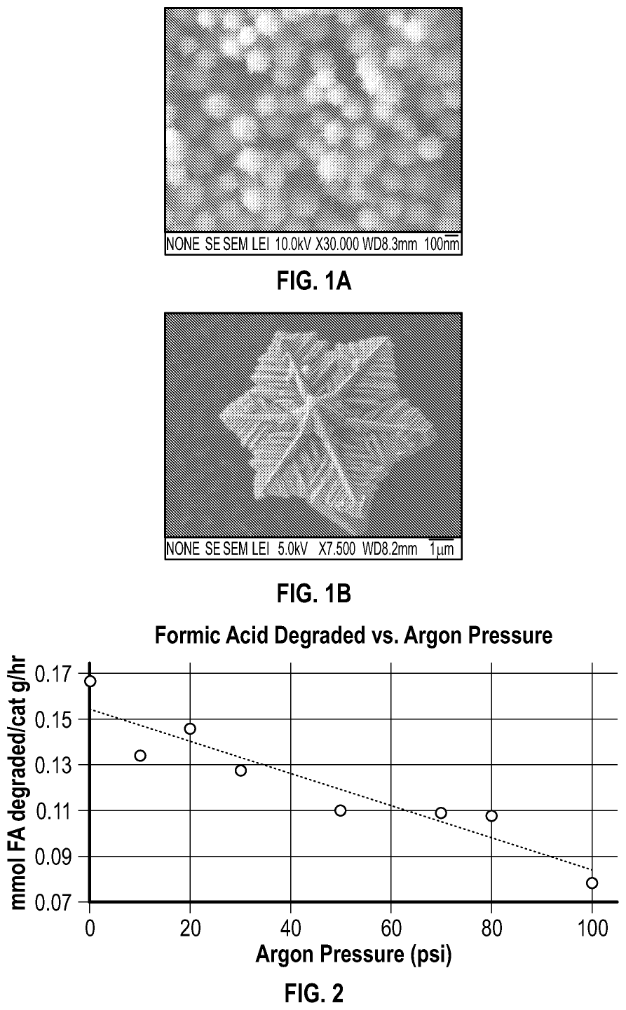

The nanostructured surface of the metal catalyst component formed through a top-down approach is different than those of a metal catalyst component formed from a bottom-up approach. As can be seen in FIGS. 1A-1B, the nanostructured surface of the metal catalyst components have a nanoleaf structure. In contrast, the nanostructured surface of the metal catalyst components of Shen, formed by acid, base, or laser etching have a nanospike or nanoflake structure. However, in some embodiments, it is possible to form the catalyst through a top-down approach where nanostructures are produced by a deposition process such as e-beam evaporation or sputter coating.

The nanostructured surface of the metal catalyst component of the instant disclosure can be formed by reacting a metal salt with a reducing agent. The metal can be any one of the metals described herein. The anion can be any suitable anion such as chloride, bromide, iodide, fluoride, nitrate, sulfide, sulfate, perchlorate and the like. In examples where the metal is cobalt, the salt can be at least one of CoCl₂, Co(NO₃)₂, CoSO₄, and mixtures thereof. The reducing agent can be one of many suitable reducing agents such as N₂H₄, N₂H₄·H₂O, NaBH₄, sodium dithionite, sodium hypophosphite, lithium aluminum hydride, tin (II) salts, hydrogen, and mixtures thereof.

After the nanostructured surface of the metal catalyst component is formed, the metal catalyst component can be “aged” for a period of time before use in the hydrogen producing reaction. The metal catalyst component can be aged for any suitable amount time. For example, the metal catalyst component can be aged for a time ranging from about 1 day to 15 days, 2 days to 10 days, less than, equal to, or greater than 1 day, 2, 3, 4, 5, 6, 7, 8, 9, or 10 days. The metal catalyst component can be aged for example in an oven under vacuum or in the presence or absence of oxygen.

Due to the production of formic acid/formate being a catalytic reduction reaction, a catalytic step to reduce the catalyst back into its initial state may be necessary. It is possible that chloride, an impurity from the catalyst synthesis, can be oxidized to chlorate and perchlorate during the reduction of CO₂.

Additionally, reactants other than chloride can be oxidized in this catalytic redox reaction. Other reactants that have been successfully oxidized during the reduction of CO₂ include urea, bromide, and iodide. Control of the oxidation process will allow the correct selection of reactant to be oxidized to increase yield and perhaps enable the collection of another value added product. For example, preliminary results demonstrating the oxidation of urea to nitrate may indicate the ability of the process to make a highly desirable byproduct like nitrate fertilizer in the course of producing hydrogen.

The amount of each component in the reaction vessel can be selected from many suitable amounts. Generally, the amount of each component can depend on the size of the reaction vessel or the amount of formic acid, hydrogen, or both to be produced.

The carbon dioxide can be supplied to the reaction vessel as a gas. The pressure of the carbon dioxide in the reaction vessel can be in a range from about 0 psi above ambient to about 150 psi.

The amount of water in the reaction vessel can be any appropriate amount.

The quantity of water is typically enough to make sure that the catalyst is submerged in the reaction mixture. While not so limited, the quantity of the water in the reaction vessel can be at least 200 μL at least about 2000 μL, at least 50 ml, at least 1 L, at least 10 L, at least 50 L or any suitable volume to conduct the reaction. The wt % of the water relative to the other components can range from about 0.01 wt % to about 60 wt %. Additionally, the water can be continuously introduced in the reaction vessel. Water can be supplied in excess of any of the aforementioned stoichiometric amounts.

In addition to the components described herein, other components can be added to the reaction vessel. It can be desirable to keep the pH within the reaction vessel above 3, above 6, above, 7, or above 9.

With all the components present and the temperature optionally increased, the reaction begins. The reaction can be allowed to proceed continuously or for any suitable finite amount of time. For example, a quantity of the metal catalyst component can be placed in a reactor. Water and carbon dioxide can be continuously supplied to the reactor. The continuous supply of water and carbon dioxide to the metal catalyst component in the reactor can produce a stream of formic acid. This process can be configured to run for any suitable amount of time. For example, the reaction can run for a time period ranging from about 0.5 hours to about 100 hours, about 20 hours to about 94 hours, about 60 hours to about 80 hours, less than, equal to, or greater than 0.5 hours, 5, 10, 15, 20, 25, 30, 35, 40, 45, 50, 55, 60, 65, 70, 75, 80, 85, 90, 95, or 100 hours. Additionally, longer times are contemplated within the scope of this disclosure (e.g., greater than 100 hours, greater than 200 hours, greater than 300 hours, greater than 400 hours, etc).

After formic acid is formed the formic acid can be stored or immediately degraded to form hydrogen. The degradation of formic acid can be conducted by contacting the formic acid with the metal catalyst component as described herein. The degradation can occur at a temperature in a range of from about 0° C. and 40° C. Conducting the degradation in the presence of light may increase the rate of degradation.

An amount of the hydrogen produced from the reaction ranges from about 20% (v/v) to about 90% (v/v), 25% (v/v) to about 35% (v/v), less than, equal to, or greater than 20% (v/v), 25, 30, 35, 40, 45, 50, 55, 60, 65, 70, 75, 80, 85, or 90% (v/v).

The methods described herein can be carried out by individually producing formic acid in one reaction vessel and then using a separate reaction vessel for the degradation of formic acid to hydrogen and carbon dioxide. In further embodiments, however, a kit can be used to produce formic acid and then degrade the formic acid to hydrogen in a single reaction vessel.

For example, a kit can include a first chamber or region that includes the metal catalyst component. The metal catalyst component can be fixed in the chamber for example in a retention device. The first chamber or region can be supplied with water and carbon dioxide as well as light. The supply of water and carbon dioxide can be continuous or segmented. Formic acid produced can be stored or sent to a second chamber or region where it reacts with the metal catalyst component to be decomposed into hydrogen. Any of the chambers can be joined to a solar panel as an example that can supply light or light energy to facilitate the respective reaction.

In some embodiments, hydrogen produced can be stored for later use. In still other embodiments, hydrogen produced can be feed directly into a system that consumes hydrogen for fuel (e.g., a hydrogen fueled gas turbine or a fuel cell stack). Formic acid produced can be stored for later degradation to hydrogen or degraded immediately to produce hydrogen for immediate use. Additionally, any remaining or produced carbon dioxide, carbonate, and/or bicarbonate can be recycled by being fed back into the system to facilitate continuous production of formic acid and/or hydrogen.

### Examples

Various embodiments of the present disclosure can be better understood by reference to the following Examples which are offered by way of illustration. The present disclosure is not limited to the Examples given herein.

### Confirmation of Results:

Hydrogen production by a Formic Acid Production Chamber has been measured on two gas chromatographs, gas chromatography—thermal conductivity detector (GC-TCD) and gas chromatography—reducing compound photometer (GC-RCP). Formic acid measurements were made with ion chromatography (IC), and gas chromatography—mass spectrometry (GC-MS). Isotope tests with D₂O and ¹³CO₂ were performed and analyzed with GC-MS. Results confirmed the formation of formic acid from water and carbon dioxide. Additional Products:

The conditions of the system determine the products formed. High temperatures form hydrocarbons, perhaps through a formic acid intermediate. Under the conditions favorable to formic acid formation, acetic acid has been detected as a product as well. It is believed that other longer chain acids may be formed.

### Catalyst Characteristics:

The surface features of the nano-structured nickel (n-Ni) and cobalt (n-Co) catalyst particles were analyzed with Scanning Electron Microscopy (SEM) and typical examples are shown in FIGS. 1A and 1B, respectively. X-Ray Diffraction (XRD) data showed face centered cubic (FCC) nickel with additional peaks for nickel hydroxide. XRD data for the cobalt particles showed a mix of approximately 76% hexagonal close packing (HCP), 19% FCC cobalt, and 5% cobalt hydroxide. X-Ray Photoelectron Spectroscopy (XPS) data for cobalt particles determined that it is covered by a thin layer of cobalt oxide, and the layer is thought to be a thickness of 10 nm or greater.

### Batch Mode Optimization Data:

### Batch Mode Optimization of Formic Acid Production:

$${H}_{2} O + {CO}_{2 ( g )} {\to }^{n - M} {HCOOH}_{( aq )}$$

(where n-M is a metal with a nanostructured surface)

Formic acid was produced with visible light using only water, an aqueous carbonate solution or CO₂, and a nickel or cobalt catalyst as shown in Table 1. Formic acid/formate can also be produced in the absence of light, however the data in Table 1 was generated using visible light irradiation. Pressure of carbon dioxide was found to have a positive effect on yield up to 50 psi. Increasing the concentration of carbonate in a non-pressurized system was found to increase the yield of hydrogen but decrease the yield of formic acid. Different types of carbonates were tested with both catalysts, and the type of carbonate was found to have a minimal effect on yield. pH tests were performed, and it is believed that increasing pH has a positive effect on yield. This could be due to an increased amount of CO₂ being dissolved in the solution and in contact with the catalyst. An outlier of this trend was found around a pH of 3.08, perhaps due to the pKa of formic acid being 3.75. Maximum lifetime batch reactor experiments were stopped at 480 hours, with the catalyst still making a yield of 2.6 μmol H₂/g cat-hr. Untreated sea water was used and was found to make 5.42 μmol H₂/g cat-hr and 0.125 μmol formic acid/g cat-hr. Methanol was also found to produce formic acid at a rate of 2.015 μmol formic acid/g cat hr. Various concentrations of methanol were tested, and this data can be seen in Table 2.

TABLE 1
Product Evolution Rates

| | | | Yield of | Yield of |
|---|---|---|---|---|
| | | | Formic Acid | Hydrogen |
| | | Time | (μmol FA/ | (μmol H₂/ |
| Catalyst | Reactant | (hr) | g cat hr) | g cat hr) |
| Nickel | K₂CO₃ | 1 | 0.119 | 0.092 |
| Cobalt | K₂CO₃ | 1 | 20.31 | 19.86 |
| Nickel/Cobalt | K₂CO₃ | 1 | 0.608 | 0.235 |
| 50:50 | | | | |
| Cobalt | 30 psi CO₂ | 2 | N/A | 35.0 |

TABLE 2
Methanol Experiments Producing Formic Acid

| Methanol % | μmoles FA formed/g cat hr |
|---|---|
| No Methanol | 2.592 |
| 1% | 2.551 |
| 20% | 2.676 |
| 50% | 3.385 |
| 75% | 2.818 |
| 100% | 2.015 |

### Batch Mode Optimization of Formic Acid (FA) Degradation by Cobalt Catalyst:

$${HCOOH}_{( aq )} {\to }^{n - M} {H}_{2 ( g )} + {CO}_{2 ( g )}$$

where n-M is n-Co or n-Ni

Pressure of argon was found to have a negative correlation with the amount of formic acid degraded, and this can be seen in FIG. 2. This is not unexpected based on the formation of gaseous products from aqueous formic acid as shown in the reaction above. It is believed temperature increases the rate of degradation. Concentration of formic acid is believed to have a first-order kinetic relationship with the degradation rate. Degradation was found to occur without sunlight, and at room temperature. Light is thought to increase the rate of degradation. Temperature was kept constant in a water bath, within a range between 25° C. and 30° C. Kinetic experiments are being performed to determine activation energy.

### Initial Data for Formic Acid Production in a Flow-Through Mode:

### Prototype Set-Up:

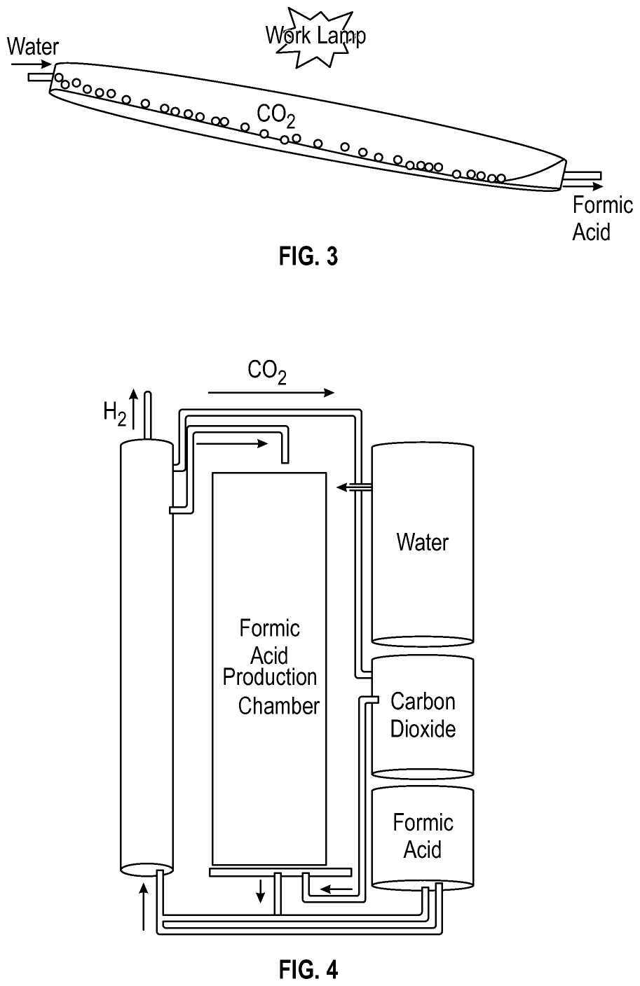

A 370 mL glass column positioned in an approximately horizontal arrangement with a slight downward angle was coated with approximately 2 g catalyst settled on the bottom (FIG. 3). The head space was pressurized with CO₂ and water was pumped into the column and allowed to flow over the catalyst bed at a rate of 1 mL/min as shown in FIG. 3. Product was collected in a graduated cylinder, and formic acid was analyzed with IC.

### Optimization Data for Flow-Through Mode Production of Formic Acid:

A potassium carbonate solution with a pressurized head of either carbon dioxide or argon gas was found to produce minimal to no formic acid in the flow-through system at a flow rate of 1 mL/min and a temperature of 50° C. In a system with water and a head space of CO₂, a temperature range from 10° C. to 70° C. was found to increase the yield of formic acid. It was found that the cobalt catalyst has reached 40 hours with no decrease in formation rate of formic acid. Data for the maximum lifetime flow-through experiments can be seen in Table 3. Maximum lifetime experiments were performed at a pressure of 90 psi, a flow rate of 1 mL/min, and a temperature of 40° C. A mixture of nickel and cobalt at a ratio of 2.5:1 was found to have the highest production rate of formic acid, with 64.8 μmol FA/g cat hr. Untreated sea water was tested with a CO₂ pressure of 83 psi, and was found to make 4.74 μmole FA/g cat hr.

TABLE 3
Cobalt Maximum Lifetime Flow Through

| Flow through run # | Total Time (hrs) | μMole FA/g cat hr |
|---|---|---|
| 1 | 8.5 | 2.426 |
| 2 | 3.5 | 8.281 |
| 3 | 8.25 | 3.714 |
| 4 | 5.5 | 2.784 |
| 5 | 7.5 | 4.413 |
| 6 | 8 | 3.811 |

### Optimization Data for Flow-Through Mode Degradation of Formic Acid:

Initial cobalt degradation flow-through experiments were performed with a 1 M formic acid solution at 20° C., with a flow rate of 5 mL/min. Hydrogen was found to be produced at 16.04 mmol/g cat hr.

### Design of Formic Acid Production Device:

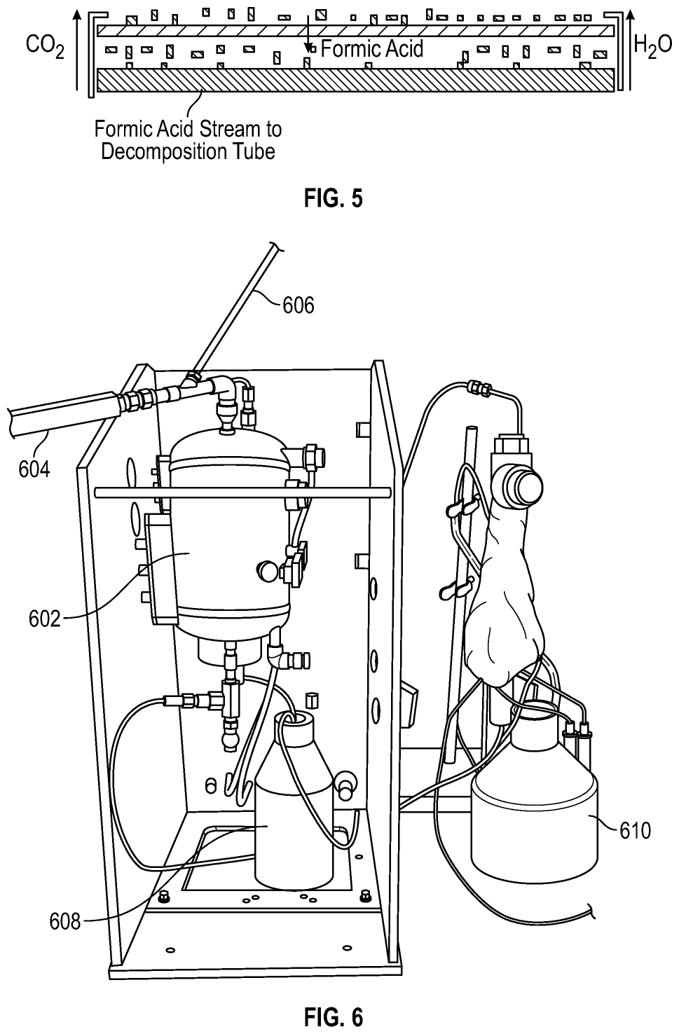

A flow-through system that produces formic acid in one chamber and then degrades it in a separate chamber is shown in FIG. 4. The catalyst is supported on a mesh screen to allow water to flow through. A side view of the formic acid production device can be seen in FIG. 5. The panel is pressurized with CO₂, and water will be dripped over the bed of catalyst and through the screen, allowing optimal residence time for formic acid production. The produced formic acid will be carried by a water stream into the degradation tube, where H₂ and CO₂ will be produced. A liquid gas separator will be used to separate gaseous products from the water stream. CO₂ and H₂ will be separated by a ceramic membrane, and the CO₂ will be introduced back into the formic acid solar panel. Excess formic acid and water will flow back into the production device to continuously collect newly produced formic acid.

### Lifetime of Catalyst:

The lifetime of the cobalt catalyst is limited by the formation of cobalt carbonate and the subsequent oxidation to CO₃O₄. Oxidation can be slowed by low temperatures. It is believed that the flow-through system will have a longer maximum lifetime because of the continuous removal of cobalt ions in the system. Cobalt carbonate is formed when the concentration of carbon dioxide and cobalt ions surpasses cobalt carbonate solubility parameters. Lifetime experiments with nickel have not been performed yet. It is believed that nickel could have a longer lifetime due to the decrease rate of oxidation of nickel. Additionally, the formation of nickel carbonate has not yet been observed during the experiments.

### Dual Chamber Apparatus

A dual chamber apparatus for the production of formic acid/formate was developed as shown in FIG. 6. As shown in FIG. 6, apparatus 600 includes vessel 602 where the catalyst is located and formic acid/formate was produced. Carbon dioxide and water are fed to vessel 602 through respective inlets 604 and 606. Formic acid/formate formed is fed to reservoir 608. Degradation products are fed to reservoir 610.

Light was not necessary to allow the reaction to proceed. Thus vessel 602 was formed from stainless steel. However, if vessel 602 was altered to allow for transmission of light therethrough, the production of formic acid/formate was increase 10-100×.

Although light could be added, it was found in some examples, that the absence of light aided in the selective formation of formic acid/formate as opposed to acetic acid. It was found that for the cobalt catalyst, that the highest selectivity of 84 moles formic acid per mole acetic acid and the highest rate of 5.8 μmol FA/g cat/min. The nickel catalyst exhibited total formic acid selectivity for the dark experiment (e.g., absence of light) at 22° C. Additionally, total selectivity of formic acid on nickel occurred for KI [0.05M], but had a significantly lower rate than the dark experiment at 22° C. It should also be recognized that while cobalt had a lower yield in the dark experiment at 10° C., a higher selectivity was observed in the dark. The higher selectivity of the thermal production of formic acid could be explained by the previously discussed temperature gradient. If the temperature is not uniform throughout the catalyst during the light assisted reactions, selectivity could be reduced due to the difference in production activation energies for formic acid and acidic acid.

It was shown that continuous feeding of water and carbon dioxide to system 600 produced adequate amounts of formic acid/formate.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed may be resorted to by those of ordinary skill in the art, and that such modifications and variations are considered to be within the scope of embodiments of the present disclosure.

### Additional Aspects.

The following exemplary aspects are provided, the numbering of which is not to be construed as designating levels of importance:
Aspect 1 provides a method of producing formic acid, hydrogen, or a mixture thereof the method comprising:
forming a reaction mixture comprising
   water, and
   carbon dioxide (for example, gaseous carbon dioxide, aqueous carbonate, aqueous bicarbonate, or a mixture thereof),
passing the reaction mixture over a metal catalyst component comprising a nanostructured surface; and
   producing a reaction product comprising formic acid, hydrogen, or a mixture thereof.
Aspect 2 provides the method of producing hydrogen of Aspect 1, further comprising contacting the reaction mixture with light.
Aspect 3 provides the method of Aspect 2, wherein the light has a wavelength in a range of from about 300 nm to about 800 nm.
Aspect 4 provides the method of any one of Aspects 1-3, wherein the metal catalyst component comprises an elemental metal or alloy thereof.
Aspect 5 provides the method of Aspect 4, wherein the elemental metal or alloy thereof ranges from about 1 wt % to about 100 wt % of the metal catalyst component.
Aspect 6 provides the method of any one of Aspects 4 or 5, wherein the elemental metal or alloy thereof ranges from about 10 wt % to about 100 wt % of the metal catalyst component.
Aspect 7 provides the method of any one of Aspects 4-6, wherein the elemental metal or alloy thereof comprises at least one of Co, Ti, Ru, Rh, Pd, Os, Ir, La, Ce, Fe, Cu, and Ni.
Aspect 8 provides the method of Aspect 7, wherein the elemental metal or alloy thereof comprises Co or Ni.
Aspect 9 provides the method of any one of Aspects 7 or 8, further comprising a support on to which the elemental metal or alloy thereof is disposed.
Aspect 10 provides the method of Aspect 9 wherein the support comprises activated carbon, aluminum oxide, or a mixture thereof.
Aspect 11 provides the method of any one of Aspects 1-10, wherein a pH of the reaction mixture is above 3.
Aspect 12 provides the method of any one of Aspects 1-11, wherein a pH of the reaction mixture is above 7.
Aspect 13 provides the method of any one of Aspects 1-12, wherein a pH of the reaction mixture is above 9.
Aspect 14 provides the method of any one of Aspects 1-13, wherein the nanostructured surface comprises a plurality of features each independently having dimensions of about 1 nm to about 1,000 nm in length, about 1 nm to about 1,000 nm in height, and about 1 nm to about 50 nm in thickness.
Aspect 15 provides the method of any one of Aspects 1-14, wherein the nanostructured surface of the metal catalyst component is formed by a method free of acid etching, base etching, laser etching, or a combination thereof.
Aspect 16 provides the method of any one of Aspects 1-15, wherein a pressure in an environment of the reaction mixture ranges from about 5 psi to about 300 psi.
Aspect 17 provides the method of any one of Aspects 1-16, wherein a pressure in an environment of the reaction mixture ranges from about 80 psi to about 175 psi.
Aspect 18 provides the method of any one of Aspects 1-17, wherein formic acid is produced and the method further comprises contacting the formic acid with the metal catalyst component to form hydrogen.
Aspect 19 provides the method of any one of Aspects 1-18, wherein a purity of the hydrogen produced ranges from about 20% (v/v) to about 90% (v/v).
Aspect 20 provides the method of any one of Aspects 1-19, wherein a purity of the hydrogen produced is about 30% (v/v).
Aspect 21 provides a kit for performing the method of any one of Aspects 1-20, the kit comprising the reaction mixture.
Aspect 22 provides the kit of Aspect 21, further comprising a source of light.
Aspect 23 provides the kit of any one of Aspects 21 or 22, wherein the metal catalyst component is located in an assembly that is configured to allow light to pass therethrough and contact the metal catalyst component.
Aspect 24 provides the kit of any one of Aspects 21-23, wherein the kit is adapted to produce formic acid and use the produced formic acid as a reactant to produce hydrogen.
Aspect 25 provides the kit of Aspect 24, wherein a first portion of the metal catalyst component is used to produce formic acid and a second portion of the metal catalyst component is used to produce hydrogen
Aspect 26 provides the kit of any one of Aspects 21-25, wherein the kit comprises a storage tank for storing formic acid, a storage tank for storing hydrogen, or both.
Aspect 27 provides the kit of any one of Aspects 21-26, wherein a purity of the hydrogen produced ranges from about 20% (v/v) to about 90% (v/v).

1. A method of producing formic acid, hydrogen, or a mixture thereof the method comprising:
forming a reaction mixture comprising
water, and
carbon dioxide comprising
gaseous carbon dioxide,
aqueous carbonate,
aqueous bicarbonate, or
a mixture thereof,
passing the reaction mixture over a metal catalyst component comprising a nanostructured surface; and
producing a reaction product comprising formic acid or a mixture of formic acid and hydrogen.

2. The method of producing hydrogen of claim 1, further comprising contacting the reaction mixture with light.

3. (canceled)

4. The method of claim 1, wherein the metal catalyst component comprises an elemental metal or alloy thereof.

5. The method of claim 4, wherein the elemental metal or alloy thereof ranges from about 1 wt % to about 100 wt % of the metal catalyst component.

6. The method of claim 5, wherein the elemental metal or alloy thereof ranges from about 10 wt % to about 100 wt % of the metal catalyst component.

7. The method of claim 4, wherein the elemental metal or alloy thereof comprises at least one of Co, Ti, Ru, Rh, Pd, Os, Ir, La, Ce, Fe, Cu, and Ni.

8. (canceled)

9. The method of claim 3, further comprising a support on to which the elemental metal or alloy thereof is disposed.

10. (canceled)

11. The method of claim 1, wherein a pH of the reaction mixture is above 3.

12. (canceled)

13. (canceled)

14. The method of claim 1, wherein the nanostructured surface comprises a plurality of features each independently having dimensions of about 1 nm to about 1,000 nm in length, about 1 nm to about 1,000 nm in height, and about 1 nm to about 50 nm in thickness.

15. The method of claim 1, wherein the nanostructured surface of the metal catalyst component is formed by a method free of acid etching, base etching, laser etching, or a combination thereof.

16. The method of claim 1, wherein a pressure in an environment of the reaction mixture ranges from about 5 psi to about 300 psi.

17. (canceled)

18. The method of claim 1, wherein formic acid is produced and the method further comprises contacting the formic acid with the metal catalyst component to form hydrogen.

19. The method of claim 1, wherein a purity of the hydrogen produced ranges from about 20% (v/v) to about 90% (v/v).

20. The method of claim 1, wherein the metal catalyst is reduced back to its initial state after production of formic acid by the oxidation of nitrogen to nitrate, chloride to chlorate, urea to nitrate, and glucose to formic acid.

21. A kit for performing the method of claim 1, the kit comprising the reaction mixture.

22. (canceled)

23. The kit of claim 21, wherein the metal catalyst component is located in an assembly that is configured to allow light to pass therethrough and contact the metal catalyst component.

24. The kit of claim 21, wherein the kit is adapted to produce formic acid and use the produced formic acid as a reactant to produce hydrogen.

25. The kit of claim 24, wherein a first portion of the metal catalyst component is used to produce formic acid and a second portion of the metal catalyst component is used to produce hydrogen

26. The kit of claim 21, wherein the kit comprises a storage tank for storing formic acid, a storage tank for storing hydrogen, or both.

27. The kit of claim 21, wherein a purity of the hydrogen produced ranges from about 20% (v/v) to about 90% (v/v).
